(19) United States
(12) Patent Application Publication
(10) Pub. No.: US 2005/0197712 A1
(43) Pub. Date: Sep. 8, 2005

(54) Prosthesis

(76) Inventors: Robert John Andrew Bigsby, Penarth (GB); Gordon William Blunn, Bassingbourn (GB); Poul T. Neilsen, Aalborg (DK); Johan Witt, Kaida Vale (GB)

Correspondence Address: HARNESS, DICKEY & PIERCE, P.L.C., P.O. BOX 828, BLOOMFIELD HILLS, MI, 48303, US

(21) Appl. No.: 11/075,178

(22) Filed: Mar. 8, 2005

(30) Foreign Application Priority Data
Mar. 8, 2004 (GB) GB0405189.2

(51) Int. Cl. A61F 2/36; A61F 2/28

(52) U.S. Cl. 623/23.27; 623/23.44

(57) ABSTRACT

A prosthesis includes a body portion adapted to engage a bone. A rod is adapted to pass through the bone. A fixing element includes a bone engaging plate and a locking piece which engages the rod and is adapted to articulate with the bone engaging plate.

### FIELD

This invention relates to prostheses and particularly but not exclusively relates to femoral hip prostheses.

### INTRODUCTION

Total hip replacement has been used in the treatment of arthritic hips since the 1960's. Femoral and acetabular prosthesis components have been used to replace defective bone tissue. Some of the most commonly used femoral components comprise a metallic stem, about 150 mm in length, which may be inserted into the intramedullary cavity, extending along the femoral axis. While this type of prosthesis has been used with success among elderly patients, the success rate among younger, more active patients tends to be lower.

As a result, a more conservative type of prosthesis has been developed, allowing a greater portion of the patient's natural bone and associated soft tissue to be preserved. In one example, during surgery, the bone may be resected on the femoral neck, just below the spherical head and the prosthesis may be mounted in the upper region of the femur, aligned with the axis of the femoral neck.

Despite the increase in success associated with conservative femoral components, attachment of the prosthesis to the bone remains a problem. In some conventional systems, bone cement, HA or press-fit designs may be used to secure the prosthesis in place. It is also known in another example to use a bolt or screw in conjunction with a fixing plate to provide greater stability, however, it has been suggested that the rigidity of these devices may cause weakening of the bone and render it less able to withstand the stresses that may be imposed when the joint is under load.

### SUMMARY OF THE INVENTION

A prosthesis includes a body portion adapted to engage a bone. A rod is adapted to pass through the bone. A fixing element includes a bone engaging plate and a locking piece which engages the rod and is adapted to articulate with the bone engaging plate.

According to various embodiments the locking piece defines a first bearing surface that is rotatable about a second bearing surface defined on the bone engaging plate. The rotation is about an axis substantially perpendicular to a longitudinal axis of the rod. The articulation of the locking piece with the bone engaging plate is spherical. The bone engaging plate includes attachment members adapted to secure the bone engaging plate to the bone. The attachment members may include screws or spikes.

A method of implanting a prosthesis into bone includes resecting a neck portion of the bone and defining a resected surface thereat. A stem is inserted into a bone tunnel formed in the bone. A collar disposed on a first end of the stem is abutted onto the resected surface. A bone engaging plate disposed on a second end of the stem is located against an outer surface of the bone. A bolt operatively coupled between the stem and the bone engaging plate is advanced relative the stem whereby spikes defined on the bone engaging plate drive into the bone surface.

According to various embodiments advancing the bolt compresses the stem and the collar into the bone. Locating the bone engaging plate against the outer surface includes locating the bone engaging plate against a lateral cortex of a femur. Advancing the bolt includes rotating a first bearing surface defined on the locking plate about a second bearing surface defined on the bone engaging plate. The rotation is about an axis substantially perpendicular to a longitudinal axis of the bolt.

Further areas of applicability of the present invention will become apparent from the detailed description provided hereinafter. It should be understood that the detailed description and specific examples, while indicating the embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, wherein:

FIG. 1 shows an exemplary embodiment of hip prosthesis;

FIG. 2 shows the positioning of the hip prosthesis of FIG. 1 within the femur;

FIG. 3 shows a hip prosthesis according to another embodiment; and

FIG. 4 shows a hip prosthesis according to another embodiment.

### DETAILED DESCRIPTION OF THE PREFERRED EMBODIMENTS

The following description of the embodiment(s) is merely exemplary in nature and is in no way intended to limit the invention, its application, or uses. Moreover, while the description below is directed to a hip prosthesis, the invention and implant may be directed to any portion of the body.

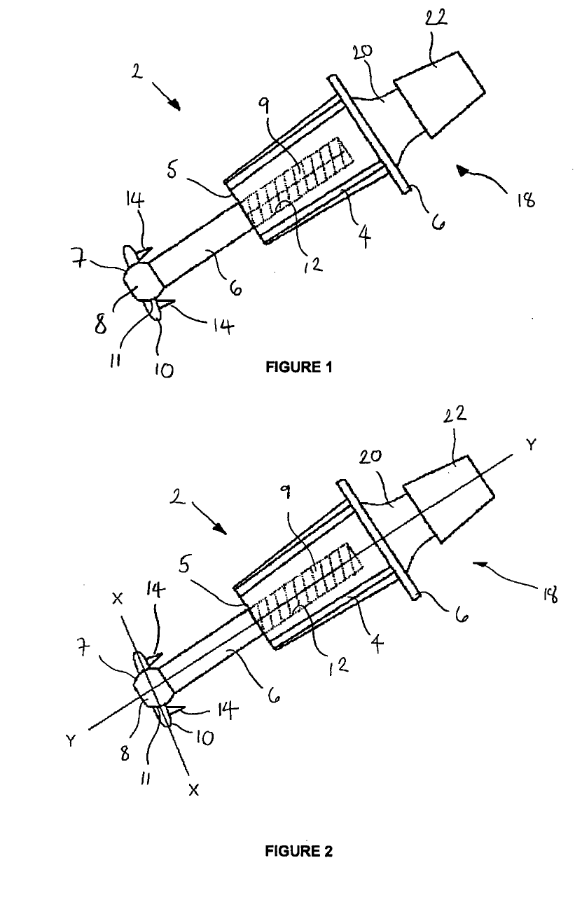

FIG. 1 shows an exemplary embodiment of hip prosthesis 2 comprising a stem 4, a bolt 6 having a head 8 and a proximal end 9, and a bone engaging plate 10. The stem 4 is tapered towards its distal end 5 and may be provided with an internally threaded bore 12 into which the end 9 of the bolt 6 can be screwed. The head 8 of the bolt 6 has a spherical bearing surface 7, which engages in a spherical opening 11 formed through the bone engaging plate 10. The bone engaging plate 10 may be free to track over the spherical bearing surface 7, so that a desired angle between a plane X-X of the bone engaging plate 10 and a longitudinal axis Y-Y of the bolt 6 can be accommodated, as shown in FIG. 2. The bearing surface 7 of the head 8 and the corresponding bearing surface of the spherical opening 11 of the bone engaging plate 10 may be formed from cobalt chrome alloy although other materials may be used.

The bone engaging plate 10 may be fitted with spikes 14, which project towards the stem 4. In an alternative embodiment, not shown, the bone engaging plate 10 could be provided with holes which receive bone screws. The outer surface of the bone engaging plate 10 may be smooth to allow free movement of soft tissues over the plate.

The stem 4 has a rounded, trapezoid shape and may be fitted with a collar 16 for abutment on a resected surface of the femoral neck. Mounted on the collar is a neck 18, comprising a support 20 and a mount 22. The mount 22 may be tapered to receive an artificial femoral head in a manner well known in the art. Those surfaces of the collar 16, stem 4 and bone engaging plate 10 which are adjacent to the bone may be provided with a porous or HA coating to promote bone ingrowth.

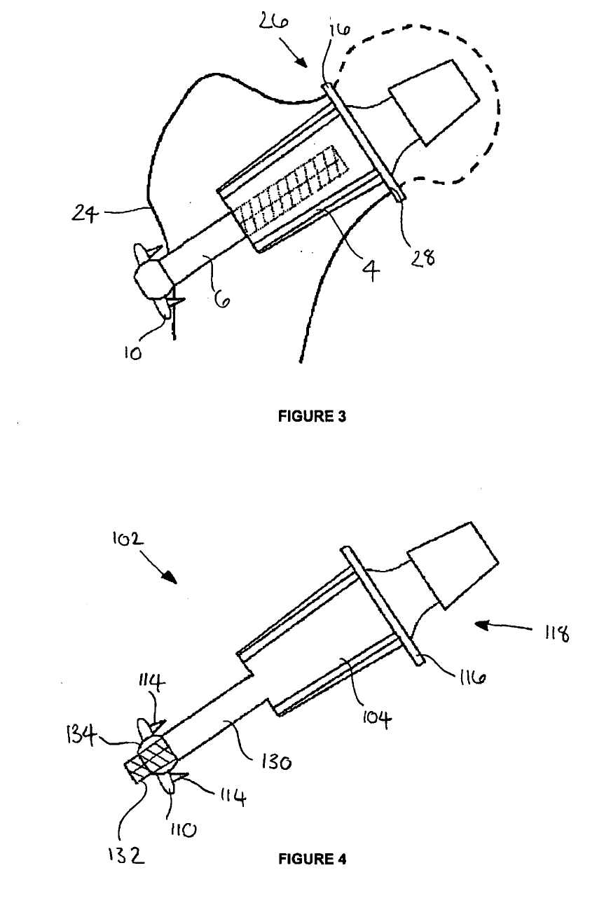

Referring to FIG. 3, the embodiment described above is shown in position within the outline of a human femur, the removed bone tissue being indicated by a dashed line. The stem 4 may be inserted into the bone, aligned with the original femoral neck 26. The collar 16 abuts the resected surface 28 of the femoral neck. The bone engaging plate 10 may be positioned on the lateral cortex 24 of the bone and the rod 6 may be passed through the lateral cortex 24. Tightening of the bolt 6 within the stem 4 drives the spikes 14 on the bone engaging plate 10 into the lateral cortex 24 and also compresses the stem 4 and collar 16 into the bone. In this manner the prosthesis may be held securely within the bone. The spherical articulation of the head 8 of the bolt 6 with the bone engaging plate 10 enables the bone engaging plate to align accurately with the lateral cortex and also enables the securely held prosthesis to maintain a certain freedom of movement, thus avoiding the disadvantages associated with the fully rigid prostheses known in the art.

Referring to FIG. 4, in an alternative embodiment, the bolt may be replaced by a rod 130 which may be attached to, or formed integrally with, the stem 104. The rod 130 has a threaded end 132, which may be adapted to receive a nut 134. The nut 134 may be substantially spherical and performs in a like manner to the head 8 of the bolt 6 in the embodiment described above. The bone engaging plate 110 is thus able to articulate on the nut 134, and thereby align accurately with the lateral cortex. The prosthesis may also be provided with a collar 116, a neck 118 and attachment spikes 114 in a manner consistent with the above description.

While the invention has been described in the specification and illustrated in the drawings with reference to various embodiments, it will be understood by those skilled in the art that various changes may be made and equivalents may be substituted for elements thereof without departing from the scope of the invention as defined in the claims. Furthermore, the mixing and matching of features, elements and/or functions between various embodiments is expressly contemplated herein so that one of ordinary skill in the art would appreciate from this disclosure that features, elements and/or functions of one embodiment may be incorporated into another embodiment as appropriate, unless described otherwise above. Moreover, many modifications may be made to adapt a particular situation or material to the teachings of the invention without departing from the essential scope thereof. Therefore, it is intended that the invention not be limited to the particular embodiment illustrated by the drawings and described in the specification as the best mode presently contemplated for carrying out this invention, but that the invention will include any embodiments falling within the foregoing description and the appended claims.

1. A prosthesis comprising:
a body portion adapted to engage a bone;
a rod which passes through the bone; and
a fixing element, comprising a bone engaging plate and a locking piece which engages the rod and is adapted to articulate with the bone engaging plate.

2. The prosthesis of claim 1 wherein the locking piece defines a first bearing surface that is rotatable about a second bearing surface defined on the bone engaging plate, and wherein the rotation is about an axis substantially perpendicular to a longitudinal axis of the rod.

3. The prosthesis of claim 2 wherein articulation of the locking piece with the bone engaging plate is spherical.

4. The prosthesis of claim 2 wherein the first and second bearing surfaces comprise a cobalt chrome alloy.

5. The prosthesis of claim 2 wherein the bone engaging plate includes attachment members adapted to secure the bone engaging plate to the bone.

6. The prosthesis of claim 5 wherein the attachment members include at least one of screws or spikes.

7. The prosthesis of claim 1 wherein the bone engaging plate defines a smooth outer surface adapted to allow free movement of soft tissue over the bone engaging plate.

8. The prosthesis of claim 1 wherein the body portion includes a stem of the prosthesis.

9. The prosthesis of claim 8 wherein the body portion is inserted into the bone in an implanted position.

10. The prosthesis of claim 9 wherein the body portion defines a substantially linear contour.

11. The prosthesis of claim 10 wherein the body portion is tapered toward a distal end.

12. The prosthesis of claim 11 wherein the body portion defines a rounded, trapezoidal shape.

13. The prosthesis of claim 12 wherein the rod is fixed to the body portion.

14. The prosthesis of claim 13 wherein the rod is integrally formed with the body portion.

15. The prosthesis of claim 14 wherein a distal end of the rod is threaded.

16. The prosthesis of claim 15 wherein the locking piece comprises a nut adapted to be engaged on the threaded end of the rod.

17. The prosthesis of claim 16 wherein the body portion defines a threaded bore.

18. The prosthesis of claim 17 wherein the rod includes a bolt and is adapted to be received in the threaded bore.

19. The prosthesis of claim 18 wherein the locking piece comprises a head of the bolt.

20. The prosthesis of claim 18 wherein the bone includes a femur and wherein the bone engaging plate is adapted to be positioned and fixed on a lateral cortex of the femur.

21. The prosthesis of claim 20 wherein the rod passes through the lateral cortex.

22. The prosthesis of claim 21 wherein the body portion is adapted to emerge from the femur at a position of a resected femoral neck.

23. The prosthesis of claim 22 wherein a proximal end of the body portion defines a neck adapted to mount a femoral head.

24. The prosthesis of claim 23 wherein the body portion defines a collar adapted to abut a resected surface of the femoral neck.

25. The prosthesis of claim 24 wherein at least one of the bone engaging plate, body portion and collar defines an outer surface having a porous coating adapted to facilitate bone ingrowth.

26. A method of implanting a prosthesis into bone comprising:
resecting a neck portion of the bone and defining a resected surface thereat;
inserting a stem into a bone tunnel formed in the bone;
abutting a collar disposed on a first end of the stem onto the resected surface;
locating a bone engaging plate disposed on a second end of the stem against an outer surface of the bone; and
advancing a bolt operatively coupled between the stem and the bone engaging plate relative the stem whereby spikes defined on the bone engaging plate drive into the bone surface.

27. The method of claim 26 wherein advancing the bolt compresses the stem and the collar into the bone.

28. The method claim 27 wherein locating the bone engaging plate against the outer surface includes locating the bone engaging plate against a lateral cortex of a femur.

29. The method of claim 28 wherein advancing the bolt includes rotating a first bearing surface defined on the locking plate about a second bearing surface defined on the bone engaging plate, and wherein the rotation is about an axis substantially perpendicular to a longitudinal axis of the bolt.
